(19) United States
(12) Patent Application Publication
(10) Pub. No.: US 2013/0289641 A1
(43) Pub. Date: Oct. 31, 2013

(54) METHOD AND SYSTEM FOR OPTIMIZING CARDIAC PACING SETTINGS

(71) Applicants: John Gustafsson, Hagersten (SE); Andreas Karlsson, Solna (SE); Andreas Blomqvist, Taby (SE); Hedberg Sven-Erik, Kungsangen (SE); Nils Holmstrom, Jarfalla (SE)

(72) Inventors: John Gustafsson, Hagersten (SE); Andreas Karlsson, Solna (SE); Andreas Blomqvist, Taby (SE); Hedberg Sven-Erik, Kungsangen (SE); Nils Holmstrom, Jarfalla (SE)

(73) Assignee: ST. JUDE MEDICAL AB, JARFALLA (SE)

(21) Appl. No.: 13/997,620

(22) PCT Filed: Dec. 23, 2010
(86) PCT No.: PCT/EP2010/070640

§ 371 Date: Jun. 24, 2013

(51) Int. Cl. A61N 1/365

(52) U.S. Cl. CPC A61N 1/36571
USPC 607/18; 607/17; 607/19

(57) ABSTRACT

The present invention relates generally to methods and systems for optimizing stimulation of a heart of a patient. Hemodynamical index signals reflecting a mechanical functioning of a heart of a patient are recorded at different hemodynamical states. Corresponding hemodynamical reference signals at corresponding hemodynamical states are recorded. At least one hemodynamical index parameter is extracted from the recorded hemodynamical index signals. The at least one hemodynamical index parameter is a measure of the mechanical functioning of the heart and a hemodynamical index model is created, wherein the hemodynamical index model is based on the at least one hemodynamical index parameter and a comparison between output results from the hemodynamical index model and corresponding hemodynamical reference signals. From this hemodynamical index model, a hemodynamical index can be derived, which then can be used in determining patient customized cardiac pacing settings of the cardiac stimulator.

### FIELD OF THE INVENTION

The present invention relates generally to methods and systems including a cardiac stimulator for optimizing stimulation of a heart of a patient.

### BACKGROUND OF THE INVENTION

Heart failure is usually a chronic, long term condition, but may occur suddenly. It may affect the left heart, the right heart, or both sides of the heart. Heart failure may be considered as a cumulative consequence of all injuries and/or stress to the heart over a person's life and the prevalence of heart failure increases constantly. For example, it is estimated that nearly 5 million people in the USA suffer from heart failure and about 400,000 new cases are diagnosed every year. The prevalence of heart failure approximately doubles with each decade of life. One of the most important means of treating heart failure is cardiac resynchronization therapy, CRT. Although CRT is a very effective way of treating heart failure in most patients there is a large percentage for which the CRT has no apparent effect at all or a limited effect.

Different estimates of the size of the so called group “non-responders” exist, but it is generally believed to be in the vicinity of 25% of all patients equipped with a CRT device. Thus, there is large portion of the patients that do not derive a clear clinical benefit (“Cardiac resynchronization in Chronic Heart Failure”, Abraham W. T., Fisher W. G., Smith, A. L. et al., New England Journal of Medicine, 2002, 346(24)).

Given that the principal mechanism of CRT is to restore cardiac synchrony, an important approach to increase clinical benefit from CRT is optimization of device programming, for example, so as to establish the AV and/or VV delays which provide maximal improvement in cardiac function. Acute improvement in systolic function has been demonstrated as a result of optimization of atrioventricular (AV) and interventricular (VV) delays at the time of CRT implantation (“Effect of pacing chamber and artioventricular delay on acute systolic function of paced patient with congestive heart failure”, Auricchio A, Stellbrink C., Block M. et al., Circulation, 1999, 99(12), and “Improved left ventricular mechanics from acute VDD pacing in patients with dilated cardiomyopathy and ventricular conduction delay”, Kass D. A., Chen C-H, Curry C., et al., Circulation, 1999, 99(12)).

Several studies have shown improvements in acute hemodynamics or myocardial efficiency as a result of device optimization (e.g. “Cardiac resynchronization with sequential biventricular pacing for the treatment of moderate-to-severe heart failure”, Leon A. R., Abraham W. T., Brozena S., et al., Journal of the American College of Cardiology, 2005, 46(12)).

A common method for adapting or optimizing the device settings for CRT is so called echo-based optimization, which may include M-mode, 2D, 3D and TDI. Echo-based optimization of the timing cycles is however time-consuming and may range from 30 minutes to two hours depending on the scope of the evaluation. Furthermore, echo-based optimization is heavily dependent on the operator, who interprets the displayed echo signals, for accuracy and consistency.

Invasive hemodynamic assessment is an accurate and reliable method but which, on the other hand, carries procedural risks and is not ideally suited for e.g. repeated optimizations to be performed at different time intervals during clinical follow-up.

Accordingly, there is a need for fast and accurate methods suitable for repeated CRT timing optimization and for patient customized CRT timing optimization.

A fast device based CRT optimization method which is suitable for repeated optimizations is St. Jude Medical's QuickOpt™ Timing Cycle Optimization, which is an algorithm that provides IEGM (Intracardiac Electrogram) based AV (Atrial-Ventricular) timing optimization in CRT and ICD (Implantable Cardioverter-Defibrillator) systems and VV (Ventricular-Ventricular) timing optimization in CRT devices in a simple and swift way. QuickOpt™ Timing Cycle Optimization is based on the hypothesis that the point of time for the closure of the Mitral valve can be estimated by measuring the interatrial conduction time (P-wave duration), that the onset of isovolumetric contraction can be measured using the peak of the R-wave and that interventricular conduction delays can be measured by evaluating simultaneous RV (Right Ventricular) and LV (Left Ventricular) IEGMs and measuring the time between the peaks of the R-waves. The goal is to characterize interatrial conduction patterns so that preload is maximized and ventricular pacing does not occur until after full closure of the mitral valve and to characterize intrinsic and paced interventricular conduction patterns so that pacing stimuli and the resultant LV and RV conduction (paced wave fronts) meet at the ventricular septum. Accordingly, QuickOpt™ Timing Cycle Optimization electrically characterizes the conduction properties of the heart to calculate optimal paced and sensed AV delay, i.e. the time interval between a paced atrial event and the ventricular impulse and a sensed atrial event and the ventricular impulse, respectively, and/or VV delay. QuickOpt™ Timing Cycle Optimization has been clinically proven to correlate with the more time-consuming echo-based methods and may be used for patients carrying CRT and dual-chamber devices at implant or follow up. QuickOpt™ Timing Cycle Optimization is an appealing optimization method since it does not require systematic measurements of a number of different AV and VV delays, which makes it very fast and simple. There are other IEGM based optimization methods among which QuickOpt™ Timing Cycle Optimization is one such method.

Despite the evident advantages of IEGM based optimization methods, such as e.g. QuickOpt™ Timing Cycle Optimization, there is an opinion within the medical community, for example, among physicians that results, e.g. timing cycles, based on input data more directly reflecting the mechanical functioning of the heart may be even more accurate and reliable.

Thus, there is still a need within the art for improved patient customized methods and systems for optimizing pacing settings including, for example, AV and VV delays for use in CRT.

### SUMMARY OF THE INVENTION

An object of the present invention is to provide improved methods for determining patient specific cardiac pacing settings of a cardiac stimulator system and improved cardiac stimulator systems capable of such determination of patient specific pacing settings for use in, for example, CRT that eliminates or at least alleviates the problems encountered with the prior art methods and systems.

A further object of the present invention is to provide improved methods for automatically optimizing patient specific cardiac pacing settings of a cardiac stimulator system for use in, for example, CRT and cardiac stimulator systems capable of such optimization that eliminates or at least alleviates the problems encountered with the prior art methods and systems.

According to a further object of the present invention, there is provided improved cardiac stimulator systems and methods for such systems capable of fast, accurate and reliable patient customized determination and optimization of pacing settings for use in, for example, CRT.

These and other objects of the present invention are achieved by means of an implantable medical device and a method having the features defined in the independent claims. Embodiments of the invention are characterized by the dependent claims.

According to an aspect of the present invention, there is provided a method for determining cardiac pacing settings of a cardiac stimulator system including a cardiac stimulator, non-implantable equipment for measurements of hemodynamical reference signals, and a number of electrodes and at least one chronically implantable hemodynamical sensor connectable to the cardiac stimulator. The method comprises recording hemodynamical index signals reflecting a mechanical functioning of a heart of a patient, wherein the hemodynamical index signals are measured by the at least one hemodynamical sensor during measurement sessions at different hemodynamical states, and recording corresponding hemodynamical reference signals reflecting a mechanical functioning of the heart measured using the non-implantable equipment, wherein the hemodynamical reference signals are measured during measurement sessions at corresponding hemodynamical states. Further, at least one hemodynamical index parameter is extracted from the recorded hemodynamical index signals, the at least one hemodynamical index parameter being a measure of the mechanical functioning of the heart and a hemodynamical index model is created, wherein the hemodynamical index model is based on the at least one hemodynamical index parameter and comparisons between output results from the hemodynamical index model and corresponding hemodynamical reference signals. From this hemodynamical index model, a hemodynamical index can be derived, which then can be used in determining patient customized cardiac pacing settings of the cardiac stimulator.

According to a second aspect of the present invention, there is provided a cardiac stimulator system including a cardiac stimulator, non-implantable equipment, and a number of electrodes and at least one chronically implantable hemodynamical sensor connectable to the cardiac stimulator. The system further comprises a data collection module of the cardiac stimulator which is configured to collect and record hemodynamical index signals reflecting a mechanical functioning of a heart of a patient, wherein the hemodynamical index signals are measured by the at least one hemodynamical sensor and/electrodes during measurement sessions at different hemodynamical states. The non-implantable equipment is configured to record corresponding hemodynamical reference signals reflecting a mechanical functioning of the heart measured using the non-implantable equipment, wherein the hemodynamical reference signals are measured during measurement sessions at different hemodynamical states. Moreover, a calculation module is configured to receive the recorded hemodynamical index signals and the recorded hemodynamical reference signals, to extract at least one hemodynamical index parameter from the recorded hemodynamical index signals for each hemodynamical state, the at least one hemodynamical index parameter being a measure of the mechanical functioning of the heart in a specific hemodynamical state and to create a hemodynamical index model, wherein the hemodynamical index model is based on the at least one hemodynamical index parameter and comparisons between output results from the hemodynamical index model and corresponding hemodynamical reference signals, wherein a hemodynamical index can be derived from the hemodynamical index model. An optimization module is configured to use the hemodynamical index model in determining timing parameter settings of the cardiac stimulator.

The present invention is based on the insight that it is possible to create a reliable and accurate hemodynamical index model that produces a hemodynamical index which can be used to determine and optimize patient specific pacing settings. This is achieved by building the model on basis of hemodynamical parameters extracted from hemodynamical index signals measured by chronically implanted sensors reflecting the patient unique mechanical functioning of the heart at specific hemodynamical states and calibrating the model against corresponding hemodynamical reference signals measured by non-implantable equipment at the same or corresponding specific hemodynamical states. Thereby, it is possible to relate the patient specific changes in hemodynamical index signal to corresponding changes in the hemodynamic reference signal. The hemodynamical index parameters are derived or extracted from hemodynamical index signals measured with chronically implanted hemodynamical sensors located in or in proximity to the heart, which thereby reflects the patient specific volumetric changes within the heart and/or related mechanical changes of the heart during the cardiac cycle in an accurate and reliable way. After a calibration of the hemodynamical model, the model can be used for ambulatory device optimization since it is possible to automatically determine and optimize patient customized settings of a cardiac stimulator using only input from the chronically implanted sensors connectable to the implanted cardiac stimulator. The hemodynamical index model can also be used for lead optimization, for example, optimization of a left ventricle lead or stimulation configuration optimization, e.g. stimulating between LV tip to a first LV ring or stimulating between a second LV ring to a third LV ring.

By creating the hemodynamical index model based on hemodynamical index parameters extracted from hemodynamical index signals obtained at different hemodynamical states, for example, at pacing using different AV and/or VV delays and/or intraventricular delays, it is possible to achieve a model that produces a hemodynamical index that can be used to, for example, optimize a pacing setting at different hemodynamical states of the heart.

According to embodiments of the present invention, the hemodynamical index model is adapted to the posture of the patient. Hence, a posture of the patient is determined and it is verified that the posture is stable. When it is verified that the body posture is stable, hemodynamical index signals reflecting a mechanical functioning of a heart of a patient at that posture is recorded. Further, corresponding hemodynamical reference signals reflecting a mechanical functioning of the heart at that posture are also recorded. Then, at least one hemodynamical index parameter is extracted from the recorded hemodynamical index signals for each hemodynamical state when the patient is in that posture and a hemodynamical index model for that posture is created. The hemodynamical index produced by this hemodynamical index model can be used in determining cardiac pacing settings of the cardiac stimulator for that specific posture.

The morphology of the recorded hemodynamical waveforms change with the posture of the patient, and this is particularly the case with regard to cardiac impedance. Therefore, the calibration of the hemodynamical index model against recorded hemodynamical reference signals will not be the same for each posture. For example, a hemodynamical index model based on hemodynamical index parameters obtained from measurements performed at supine cannot be calibrated against hemodynamical reference signals obtained by measurements at a standing position of the patient without an impaired accuracy of the hemodynamical index. The process of finding a hemodynamical index model capable of producing an accurate and reliable hemodynamical index must therefore be repeated for every used posture, e.g. supine and standing up. According to embodiments of the present invention, one hemodynamical index model is created for a specific posture of the patient. It is thus possible to create one hemodynamical model for each body posture. During, for example, an optimization of pacing settings by, for example, variation of AV- and VV delays over predetermined ranges, the posture at which the optimization is performed should therefore be determined.

According to embodiments of the present invention, the hemodynamical index model is adapted to the activity level of the patient. This can be combined with a posture adapted model. Thus, it is possible to create a model adapted for a specific body posture and a specific activity level, for example, a model adapted for supine and rest. According to embodiments, one hemodynamical index model is created for one body posture and one activity level, and the derived parameter is adapted with predetermined values if the patient is in other postures and/or activity levels.

In order to create a hemodynamical index model for a specific activity level, an activity level of the patient is first determined and it is verified that the activity level is stable. Thereafter, hemodynamical index signals reflecting a mechanical functioning of a heart of a patient at the activity level are recorded and corresponding hemodynamical reference signals reflecting a mechanical functioning of the heart at the activity level are recorded. At least one hemodynamical index parameter is extracted from the recorded hemodynamical index signals for each hemodynamical state when the patient is at the activity level. The hemodynamical index model is created for the activity level and can be used to produce a hemodynamical index for determining cardiac pacing settings of the cardiac stimulator for the activity level.

According to embodiments of the present invention, an optimization of timing parameters of a cardiac stimulator of the patient using the hemodynamical index comprises:

performing a sweep over a predetermined number of different timing parameter settings of the cardiac stimulator, each timing parameter setting resulting in a specific hemodynamical state of the heart;

recording at least one hemodynamical index signal for each timing parameter setting;

extracting at least one hemodynamical index parameter from the recorded at least one hemodynamical index signal for each timing parameter setting;

deriving a hemodynamical index using the hemodynamical index model for each timing parameter setting; and

selecting timing parameter settings corresponding to the maximal hemodynamical index for the cardiac stimulator.

Depending on the number of timing parameters different approaches for the timing parameter optimization can be used. For example, a so called full grid search can be performed, which means that each possible combination of timing parameters is investigated. Hence, one hemodynamical index is derived for each possible combination of timing parameters, for example, in the case of an optimization of AV and VV delays each combination of AV and VV delays are investigated. For each additional parameter in such a search pattern, one dimension is added to the search grid. A less time consuming approach is a so called cross search. This means that one parameter is first varied over the different parameter values with the second parameter or the other parameters unchanged. Thereafter, when an optimal parameter value has been found for the first parameter, the second parameter is varied while keeping the first parameter unchanged at the optimal value (in case of two parameters). For example, an AV delay is first varied while keeping the VV delay constant. When the optimal AV delay has been found the search for an optimal VV delay is initiated. This cross search can be improved by using multiple iterations, e.g. after an optimal VV delay has been found the AV delay search is restarted, this time with the optimal VV delay from the first iteration.

According to embodiments of the present invention, timing parameters that can be optimized comprises atrioventricular (AV) delay, interventricular (VV) delay, intraventricular delay (e.g. using a first LV ring to a second LV ring unipolar pacing delay or LV tip—a first LV ring to a second LV ring—a third LV ring bipolar pacing delay). Further, timing parameters that can be optimized comprises e.g. interatrial delay or left atrial to LV tip—LV ring delay. The timing parameters mentioned above is a non-exhaustive list of possible timing parameters that can be optimized using the present invention.

The hemodynamical index model can be based on one or more hemodynamical index parameters extracted from one or several different hemodynamical index signals. By using parameters from several signals it is possible to create a hemodynamical index model that produces a broader picture of the hemodynamics of the heart. For example, pressure signals obtained in the cardiovascular system can be used as hemodynamical index signals. A non-exhaustive list is given below:

Left atrial pressure (LAP)

Left ventricular pressure (LVP)

Aortic pressure (AoP)

Central venous pressure (CVP

Right atrial pressure (RAP)

Right ventricular pressure (RVP)

Pulmonary artery pressure (PAP)

Such pressure signals can be measured using chronically implanted pressure sensors. Another conceivable hemodynamical signal suitable as hemodynamical index signal is heart sound signals measured, for example, using a 3D sensor (e.g. accelerometer or microphone) arranged in the can of the cardiac stimulator. This 3D sensor may also be used to determine the body posture. Further, a photoplethysmograph signal (PPG) can be used as hemodynamical index signal. Yet another conceivable hemodynamical signal that may be used as hemodynamical index signal is a lead accelerometer signal. Movement and acceleration of heart wall during the heart cycle deliver a continuous signal that may be used for timing optimization. For example, a tip accelerometer may be used for this purpose (Bordachar P., “Hemodynamic assessment of right, left and biventricular pacing by peak endocardial acceleration and echography in patient with end-stage heart-failure”, Pacing Clin Electrophysiol 2000; 23: 1726-30). Cardiac impedance has shown to be an accurate hemodynamical measure. A non-exhaustive list of examples of suitable electrode configurations include
Current injection between a first LV ring and RV ring and voltage sensing between a second LV ring and RV coil. Impedance measurements using this electrode configuration main reflects left and right ventricle hemodynamics.
Current injection between RA tip and RA ring and voltage sensing between RA tip and RA ring. Impedance measurement using this electrode configuration mainly reflects right atrium hemodynamics.
Current injection between RV tip and RV ring and voltage sensing between RV tip and RV ring. Impedance measurement using this electrode configuration mainly reflects right ventricle hemodynamics.
Current injection between LV tip and LV ring and voltage sensing between LV tip and LV ring. Impedance measurement using this electrode configuration mainly reflects left ventricle hemodynamics

A number of different impedance parameters can be obtained or determined based on the impedance signals. For example, from cardiogenic impedance signals, for example, peak-to-peak (p2p), slope, fractionation, diastolic dispersion, or average value may be extracted. Further, it is possible to extract or determine, for example, phase angle, imaginary part, or magnitude of the impedance.

According to embodiments of the present invention, the hemodynamical reference signals are measured using non-implantable equipment including (non-exhaustive) for example:
Equipment for echocardiography: For example, for AV delay optimization it is conceivable to use LV dP/dtₘₐₓ or LV outflow tract VTI or Myocardial performance index as hemodynamical reference signal and for VV delay optimization is conceivable to measure interventricular dyssynchrony or time to peak systolic velocity at TDI as hemodynamical reference signal.
An invasive LV pressure catheter: For AV and VV delay optimization is conceivable to use, for example, LV dP/dtₘₐₓ as hemodynamical reference signal.
Equipment for finger photoplethysmography: For AV and VV delay optimization it is conceivable to use, for example, systolic blood pressure (SBP) or pulse pressure (PP) as hemodynamical reference signal.
Equipment for acoustic cardiography: For AV and VV delay optimization it is conceivable to use, for example, electromechanical activation time (EMAT) as hemodynamical reference signal.
Equipment for impedance cardiography: For AV and VV delay optimization it is conceivable to use, for example, stroke volume (SV) or cardiac output (CO) as hemodynamical reference signal.
Equipment for radionuclide ventriculography: For AV and VV delay optimization it is conceivable to use, for example, left ventricular ejection fraction (LVEF) as hemodynamical reference signal.
Equipment for surface ECG: For VV delay optimization it is conceivable to use, for example, QRS duration as hemodynamical reference signal.

According to embodiments of the present invention, the hemodynamical index model can be adapted to a specific heart rate or heart rate range. For that purpose, the hemodynamic index signals and the hemodynamic reference signals are measured at that specific heart rate or heart rate range at the different combinations of timing parameters, for example, at different combinations of AV and VV delays. A hemodynamic index model adapted for a specific heart rate or heart rate range can be used to optimize timing parameter setting for that particular heart rate range or heart rate. The hemodynamical index signals and hemodynamical reference signals can be measured at different intrinsic heart rates or heart rate ranges or at different programmed base rates. This entail rate adaptive optimized settings.

Further objects and advantages of the present invention will be discussed below by means of exemplifying embodiments.

These and other features, aspects and advantages of the invention will be more fully understood when considered with respect to the following detailed description, appended claims and accompanying drawings.

### BRIEF DESCRIPTION OF THE DRAWINGS

Exemplifying embodiments of the invention are illustrated by way of example and not by way of limitation in the figures of the accompanying drawings in which like references indicate similar elements. It should be noted that references to “an” or “one” embodiment in this discussion are not necessarily to the same embodiment, and such references mean at least one.

FIG. 1 is a schematical diagram illustrating a system in which the method according to the present invention can be implemented.

FIG. 2 is a schematical view illustrating a cardiac stimulator, in which the present invention may be implemented, connected to a heart via medical leads.

FIG. 3 is a schematical view illustrating details of the cardiac stimulator shown in FIG. 2.

FIG. 4 a flow chart illustrating steps of the method according to embodiments of the present invention.

FIG. 5 is a flow chart illustrating steps of the method according to embodiments of the present invention.

FIGS. 6a-6d are diagrams showing recorded cardiogenic impedance waveforms.

FIG. 7 is a diagram illustrating an example of a comparison between hemodynamical reference signals and hemodynamic indices derived from a hemodynamical model.

FIG. 8 is a flow chart illustrating steps of the method according to embodiments of the present invention.

### DESCRIPTION OF EXEMPLIFYING EMBODIMENTS

The following is a description of exemplifying embodiments in accordance with the present invention. This description is not to be taken in limiting sense, but is made merely for the purposes of describing the general principles of the invention. It is to be understood that other embodiments may be utilized and structural and logical changes may be made without departing from the scope of the present invention.

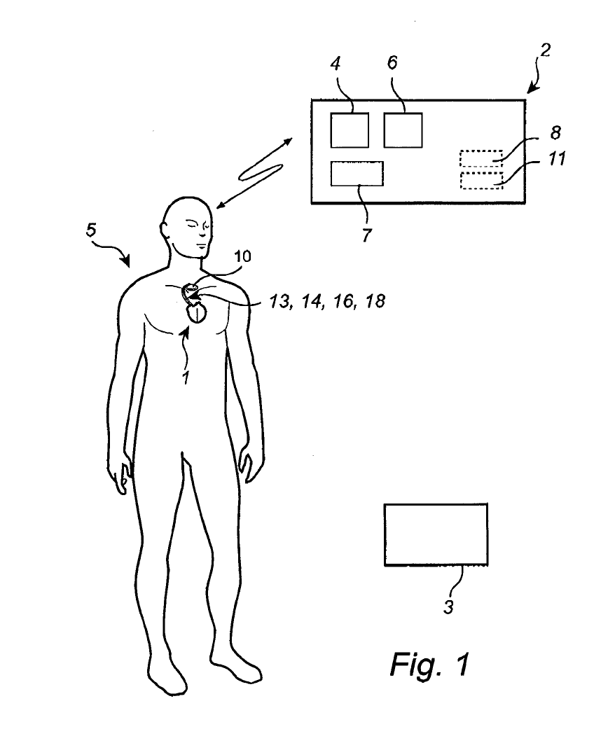

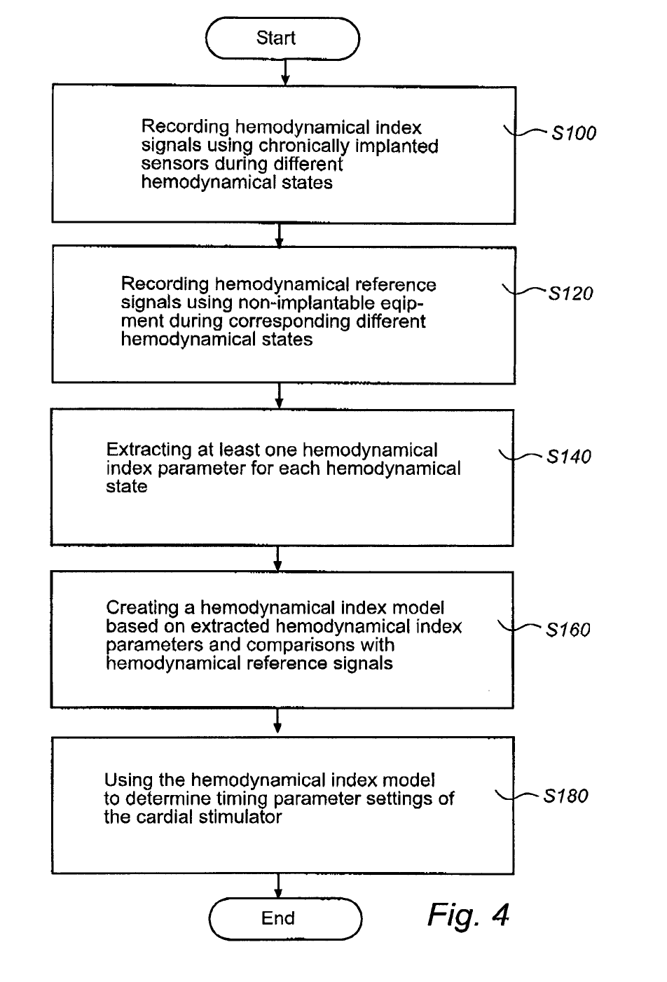

Referring to FIG. 1, a system environment in which the method according to the present invention may be implemented will be described. Different embodiments of the method according to the present invention are briefly described with reference to FIG. 1-3 but will be described in more detail with reference to FIGS. 4-5 and 8.

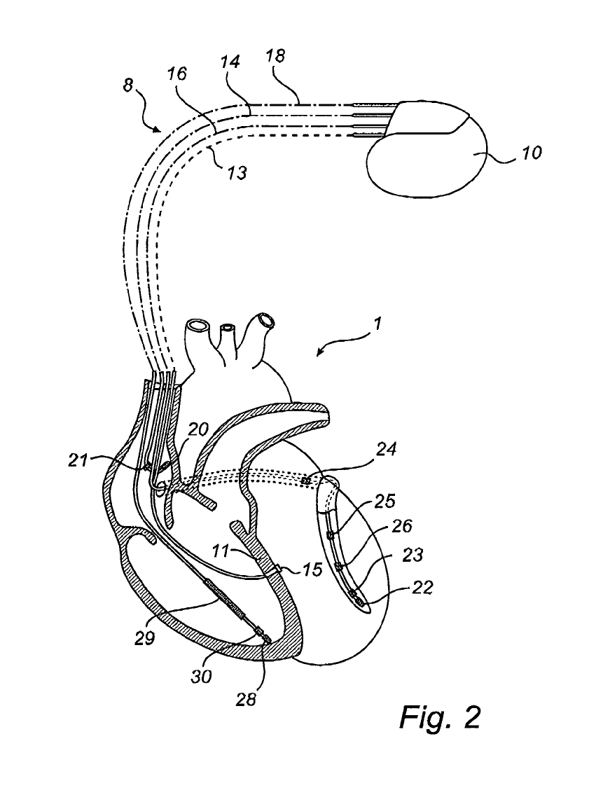

According to the present invention, a hemodynamical index model is created based on measurements of hemodynamical index signals reflecting the mechanical work of a heart 1 using chronically implanted sensors, for example, a chronically implanted pressure sensor for measuring the left ventricular pressure (see FIG. 2), connectable to a cardiac stimulator 10. The hemodynamical index model is calibrated against corresponding hemodynamical reference signals captured by non-implantable equipment 3 simultaneously to the hemodynamical index signals or at least during corresponding hemodynamical conditions of the heart 1. After calibration, the hemodynamical index model can be utilized to automatically determine and optimize cardiac pacing settings of a cardiac stimulator 10 using only input from the chronically implanted sensors connectable to the cardiac stimulator 10.

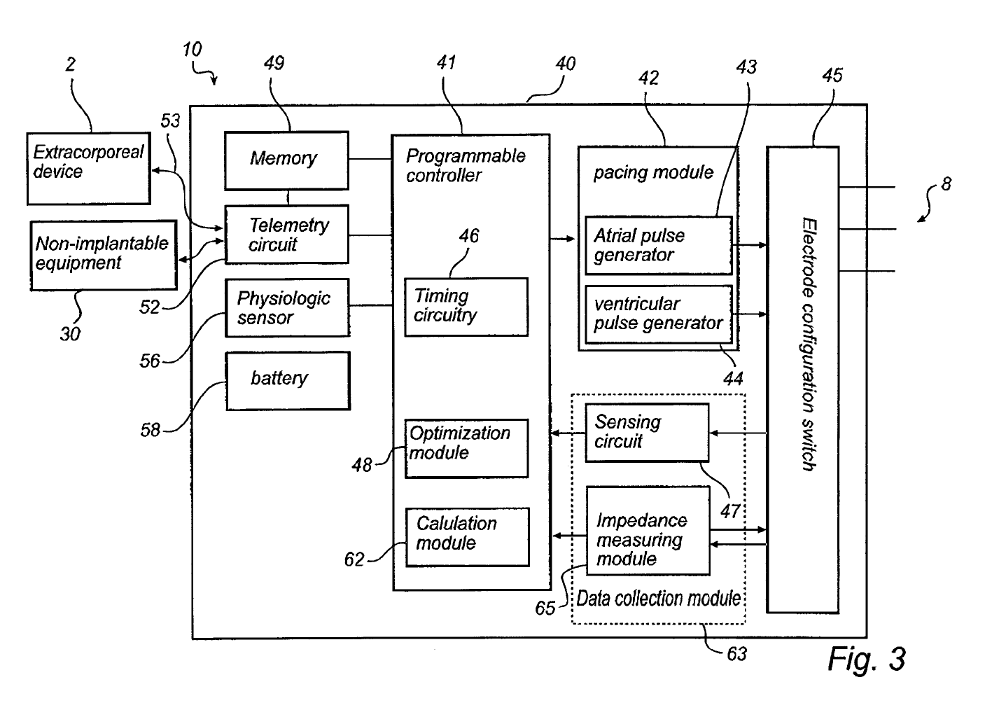

The cardiac stimulator 10 is connectable to one or more medical leads 13, 14, 16, and/or 18 including electrodes and/or sensors, for example, a left ventricle pressure sensor 15 arranged in a lead 13 (see FIG. 2). The implantable cardiac stimulator 10 can thereby be set in electrical communication with the patient's heart 1 by way of the leads 13, 14, 16, and/or 18, for example, suitable for delivering multichamber stimulation therapy, for example, via a right ventricle lead 18, a right atrium lead 14, and a left ventricle lead 16 (see FIG. 2). By using electrodes of the medical leads 8 and/or the housing or can 40 (see FIG. 3) of the cardiac stimulator 10, it is also possible to, for example, measure intracardiac impedance of the heart 1. As shown in FIG. 2, the cardiac stimulator 10 is coupled to an implantable right atrial lead 14 having, for example, an atrial tip electrode 20, which typically is implanted in the patient's right atrial appendage or septum. FIG. 2 shows the right atrial lead 14 as also having an atrial ring electrode 21. Thereby, the cardiac stimulator is capable of sensing atrial signals and providing right atrial chamber stimulation therapy. To sense left atrial and ventricular cardiac signals and to provide left chamber pacing therapy the cardiac stimulator 10 is coupled to a coronary sinus lead 16 designed for placement in the coronary sinus region via the coronary sinus for positioning a distal electrode adjacent to the left ventricle and/or additional electrode(s) adjacent to the left atrium.

As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible via the coronary sinus.

The lead 16 is designed to receive left atrial and ventricular cardiac signals and to deliver left ventricular pacing therapy using, for example, a left ventricular tip electrode 22, a first, a second, and a third left ventricular ring electrode 23, 26, and 25, and left atrial pacing therapy using, for example, a left atrial ring electrode 24.

The cardiac stimulator 10 is also in electrical communication with the heart 1 by way of an implantable right ventricular lead 18 having, in this embodiment, a right ventricular tip electrode 28, a right ventricular coil 29 and a right ventricular ring electrode 30. Typically, the right ventricular lead 18 is transvenously inserted into the heart 1 to place the right ventricular tip electrode 28 in the right ventricular apex. The right ventricular lead 18 is capable of sensing or receiving cardiac signals, and delivering stimulation in the form of pacing therapy.

As briefly mentioned above, the cardiac stimulator 10 may also be connectable to chronically implanted hemodynamical sensors, for example, a hemodynamical sensor 15, which in one particular embodiment of the present invention is a pressure sensor 15 attached to septum 11 arranged in a medical lead 13 coupled to the cardiac stimulator 10. A suitable pressure sensor is, for example described, in the co-pending application PCT/EP2010/058624 by the same applicant, U.S. Pat. No. RE 39,863, U.S. Pat. No. 6,248,083, or U.S. Pat. No. RE 35,648, herein incorporated by reference.

However, other types of hemodynamical sensors may alternatively or as a complement be used including accelerometers for measuring pressure changes in the left ventricle, flow probes, load indicators for measuring geometrical changes in the cardiac tissue e.g. in septum, heart sound sensors, or photoplethysmographic sensors.

Furthermore, the non-implantable equipment 3 is capable of measuring hemodynamical reference signals of the heart 1 of the patient 5 and may be configured to communicate with a programmer unit 2 and with the cardiac stimulator 10, e.g. wirelessly using telemetry, for example, so as to transmit measurement data including measured hemodynamical reference signals. The non-implantable equipment 3 is not intended for chronic implantation and may include at least one or a combination of the equipment presented below. The list of example equipment given below is non-exhaustive. Further, examples of hemodynamical reference signals measured with respective equipment are also given together with examples of suitable timing parameter that can be varied to create the hemodynamical index model:
Equipment for non-invasive echocardiography: This equipment may be based on ultrasound crystals generating pulses from a hand held probe that reflects tissue properties. For example, for AV delay optimization it is conceivable to use LV dP/dtₘₐₓ or LV outflow tract VTI or Myocardial performance index as hemodynamical reference signal and for VV delay optimization is conceivable to measure interventricular dyssynchrony or time to peak systolic velocity at TDI as hemodynamical reference signal. By automatically moving the ultrasound beam, a 2D picture can be produced from which parameters can be derived. Examples of such parameters includes
   Aortic velocity time integral (correlates with stroke volume (SV))
   Transmitral flow which can be used for AV optimization by studying the A- and E-waves and their relationship.
   Intra- and inter ventricular dyssynchrony for VV-optimization (e.g. septal to lateral wall activation delay)
An invasive LV pressure catheter: For AV and VV delay optimization is conceivable to use, for example, LV dP/dtₘₐₓ as hemodynamical reference signal.
Equipment for finger photoplethysmography: For AV and VV delay optimization it is conceivable to use, for example, systolic blood pressure (SBP) or pulse pressure (PP) as hemodynamical reference signal.
Equipment for acoustic cardiography: For AV and VV delay optimization it is conceivable to use, for example, electromechanical activation time (EMAT) as hemodynamical reference signal.
Equipment for impedance cardiography: For AV and VV delay optimization it is conceivable to use, for example, stroke volume (SV) or cardiac output (CO) as hemodynamical reference signal.
Equipment for radionuclide ventriculography: For AV and VV delay optimization it is conceivable to use, for example, left ventricular ejection fraction (LVEF) as hemodynamical reference signal.
Equipment for surface ECG: For VV delay optimization it is conceivable to use, for example, QRS duration as hemodynamical reference signal.
Swan-Ganz catheter: This technique measures cardiac output by monitoring temperature changes after bolus injection of a cold liquid. The injection is made into a proximal port of a catheter containing a thermistor mounted at the tip, which normally is placed in the pulmonary artery. The thermistor measures the sequential changes in temperature over time. The cardiac output is inversely related to the area under a plotted curve over the thermodilution. This is a standard method for monitoring cardiac output. Based on the measured cardiac output, stroke volume can be determined by dividing the cardiac output with heart rate. The stroke volume can then be used to optimize the AV and/or VV delays.
Catheter based equipment for measuring pulmonary capillary wedge pressure (left atrial pressure surrogate): Often a balloon-tipped, multi-lumen catheter (Swan-Ganz catheter) is inserted into a peripheral vein, advanced into right atrium, right ventricle, pulmonary artery and into a branch of the pulmonary artery. Behind the tip of the catheter, a small balloon is arranged, which may be inflated with air. The catheter has a first port at the distal tip and a second port placed more proximally relative to the balloon. The ports are connected to pressure transducers. When placed in a branch of the pulmonary artery, the distal port measures pulmonary artery pressure and the proximal port measures right atrial pressure. The balloon is then inflated, which occludes the branch of the pulmonary artery. When this occurs, the pressure in the distal port rapidly falls, and after a period of time, reaches a stable lower level that is similar to the left atrial pressure. Thereafter, the balloon is deflated. This catheter can also be used to measure cardiac output.
Millar catheter or Radi pressure wire for measuring left ventricular pressure: For example, the left ventricular pressure can be measured by catheterization of the left ventricle e.g. through the femoral artery and a retrograde passage through the aortic valve. Using the left ventricle pressure signals, LV dP/dtₘₐₓ can be achieved. In order to optimize AV and/or VV delays, LV dP/dtₘₐₓ can be maximized.
   The non-implantable equipment 3 is used to measure and record at least one hemodynamical signal reflecting the volumetric changes and related mechanical work of the heart 1. As will be described below, these recorded hemodynamical signals are used to calibrate the hemodynamical index model. That is, the results from the hemodynamical index model are verified against corresponding hemodynamical signals.

Furthermore, the cardiac stimulator 10 may be configured to communicate with the extracorporeal equipment, such as a programmer unit or workstation 2. The programmer unit 2 may comprise a control unit 4, a memory unit 6, communication unit (e.g. a telemetry unit) 7, and a display unit (not shown). A physician may, for example, initiate an optimization of timing parameter settings of the cardiac stimulator 10 via the programmer unit 2 e.g. at a follow-up visit. Further, the programmer unit 2 may include a calculation module 8 and an optimization module 11, which are described in more detail below with reference to FIG. 3. The methods described herein may thus be executed in the programmer unit 2 and the cardiac stimulator 10, for example, an optimization procedure using a created hemodynamical model can be executed in the programmer unit 2 and the cardiac stimulator 10, or only in the cardiac stimulator 10.

In FIG. 3, an exemplary, simplified block diagram depicting various components of the cardiac stimulator according to embodiments of the present invention is shown. The cardiac stimulator 10 is capable of delivering cardiac resynchronization therapy and is configured to integrate both monitoring and therapy features, as will be described below. The cardiac stimulator 10 collects and processes data about the heart 1 from one or more sensors. Further, the cardiac stimulator 10 collects and processes data about the heart 1 from electrode pairs for sensing cardiac electrogram (EGM) signals. While a particular multi-chamber device is shown, it is to be appreciated and understood that this is done for illustration purposes only. Thus, the techniques and methods described below can be implemented in connection with any suitable configured or configurable stimulation device. Accordingly, one of skill in the art could readily duplicate, eliminate, or disable the appropriate circuitry in any desired combination to provide a device capable of treating the appropriate chamber with pacing stimulation including cardiac resynchronisation therapy.

The cardiac stimulator 10 has a housing 40, often referred to as the “can” or “case electrode”. The housing 40 may function as a return electrode in “unipolar” modes. Further, the housing 40 includes a connector (not shown) having a plurality of terminals (not shown) for connection with electrodes and/or sensors.

The cardiac stimulator 10 includes a programmable microcontroller or control module 41 that inter alia controls the various modes of stimulation therapy. As well known within the art, the microcontroller 41 typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 41 includes the ability to process or monitor input signals (data or information) as controlled by a program stored in a designated block of memory. The type of microcontroller is not critical to the described implementations. Rather, any suitable microcontroller 41 may be used that carries out the functions described herein. The use of micro-processor based control circuits for performing timing and data analysis are well known in the art.

Furthermore, the cardiac stimulator 10 includes pacing module 42 adapted to provide pacing signals for delivery to the patient. The pacing module 42 comprises an atrial pulse generator 43 and a ventricular pulse generator 44 that generate pacing stimulation pulses for delivery by the leads 8 via an electrode configuration switch 45. It is understood that in order to provide stimulation therapy in each of the four chambers, the atrial and ventricular pulse generators 43 and 44, may include dedicated, independent pulse generators, multiplexed pulse generators, or shared pulse generators. The pulse generators 43 and 44 are controlled by the microcontroller 41 via appropriate control signals to trigger or inhibit stimulation pulses.

The microcontroller 41 further includes timing control circuitry 46 to control timing of the stimulation pulses (e.g. pacing rate, AV delay, VV delay, etc.) as well as to keep track of timing of refractory periods blanking intervals, etc. which is well known in the art. In addition, the microcontroller 41 may include components such as e.g. an arrhythmia detector (not shown) and/or a heart signal morphology detector (not shown).

The aforementioned components may be implemented as part of the microcontroller 41, or as software/firmware instructions programmed into the device and executed on the microcontroller 41 during certain modes of operation.

A data collection module 63 is configured to collect and record hemodynamical index signals reflecting a mechanical functioning of a heart of a patient measured by at least one hemodynamical sensor and/electrodes during measurement sessions at different hemodynamical states, for example, a left ventricle pressure sensor 15 (see FIG. 2). The data collection module may comprise a sensing circuit 47 comprising atrial sensing circuits and ventricular sensing circuits may also be coupled to the leads 8 through the switch 45 for detecting the presence of cardiac activity in each of the four chambers of the heart. Accordingly, the atrial sensing circuits and ventricular sensing circuits 47 may include dedicated sense amplifiers, multiplexed amplifiers, or shared amplifiers. The sensing circuit 47 may also be configured to receive input data signals from, for example, the left ventricle pressure sensor 15. The data collection module 63 may also comprise an impedance measuring module 65, including e.g. a current generating circuit and voltage sensing circuit. A non-exhaustive list of examples of suitable electrode configurations for measuring cardiogenic impedance includes with reference to FIG. 2.
Current injection between the first LV ring 23 and RV ring 30 and voltage sensing between the second LV ring 26 and RV coil 29. Impedance measurements using this electrode configuration mainly reflects left and right ventricle hemodynamics.
Current injection between RA tip 20 and RA ring 21 and voltage sensing between RA tip 20 and RA ring 21. Impedance measurement using this electrode configuration mainly reflects right atrium hemodynamics.
Current injection between RV tip 28 and RV ring 30 and voltage sensing between RV tip 28 and RV ring 30. Impedance measurement using this electrode configuration mainly reflects right ventricle hemodynamics.
Current injection between the first LV ring 23 and the second LV ring 26 and voltage sensing between the first LV ring 23 and the second LV ring 26. Impedance measurement using this electrode configuration mainly reflects left ventricle hemodynamics

In embodiments of the present invention, a calculation module 62 and an optimization module 48 are included in the cardiac stimulator 10, and, preferably implemented in the controller 41.

The calculation module 62 is, for example, configured to extract at least one hemodynamical index parameter from recorded hemodynamical index signals for each hemodynamical state. Further, the calculation module 62 may be configured to create a hemodynamical index model, wherein the hemodynamical index model is based on the at least one hemodynamical index parameter and a comparison between output results from the hemodynamical index model and corresponding hemodynamical reference signals. For this purpose, the hemodynamical reference signals have to be transferred to the cardiac stimulator from the non-implantable equipment 3 wirelessly via the telemetry circuit 52. Hemodynamical indices can be derived from the created hemodynamical index model for different hemodynamical states, which indices can be used for optimizing the timing parameter setting of the cardiac stimulator 10. As mentioned above, the calculation module may be implemented in the programmer unit 2.

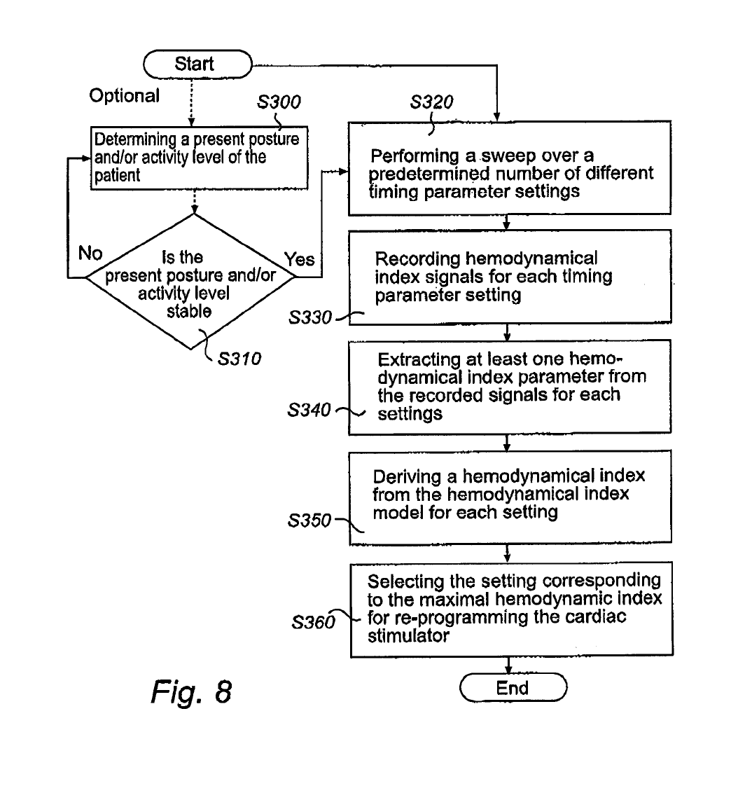

The optimization module 48 is configured to optimize timing parameter settings of the cardiac stimulator 10 using the hemodynamical index model, for example, AV and VV delays. According to an embodiment of the present invention, the optimization module 48 is configured to execute the following steps (which also are described below with reference to FIG. 8):
Instructing the pacing module 42 and timing control circuitry 46 to perform a sweep over a predetermined number of different timing parameter settings of the cardiac stimulator. For example, +/−10 to 40 ms around the currently programmed delays in case AV and VV delays are to be optimized.
Instruct the data collection module 63 to record hemodynamical index signals for each timing parameter setting.
Instruct the calculation module 62 to extract at least one hemodynamical index parameter from the recorded hemodynamical index signals for each timing parameter setting.
Input the extracted hemodynamical index parameters in the hemodynamical index model and derive a hemodynamical index using the hemodynamical index model for each timing parameter setting.
Select the timing parameter setting that corresponds to the maximal hemodynamical index and instruct the pacing module 42 and timing control circuitry 46 to use that timing parameter setting for pacing of the heart 1.

The output from the atrial sensing circuits and ventricular sensing circuits 47 are connected to the microcontroller 41, which, in turn, is able to control the atrial sensing circuits and ventricular sensing circuits 47.

Furthermore, the microcontroller 41 is coupled to a memory 49 by a suitable data/address bus (not shown), wherein the programmable operating parameters used by the microcontroller 41 are stored and modified, as required, in order to customize the operation of the cardiac stimulator to the needs of a particular patient. Such operating parameters define, for example, pacing pulse amplitude, pulse duration, etc. Advantageously, the operating parameters may be non-invasively programmed into the memory 49 through a communication module 52 including, for example, a telemetry circuit for telemetric communication via communication link 53 with the programmer unit 2 or a diagnostic system analyzer. The telemetry circuit advantageously allows intracardiac electrograms and status information relating to the operation of the device 10 to be sent to the programmer unit 2 through an established communication link 53.

The cardiac stimulator 10 may further include a physiologic sensor 56, commonly referred to as a “rate-responsive” sensor because it is typically used to adjust pacing stimulation rate according to the exercise state of the patient. While shown as being included within the stimulator 10, it is to be understood that the physiologic sensor 56 also may be external to the stimulator, yet still be implanted within or carried by the patient. Examples of physiologic sensors include sensors that, for example, sense respiration rate, or activity variance.

Moreover, the cardiac stimulator 10 additionally includes a battery 58 that provides operating power to all of the circuits shown in FIG. 3. Preferably, the stimulator 10 employs lithium or similar battery technology.

As mentioned above, the cardiac stimulator 10 is connectable to one or several chronically implantable hemodynamical sensors. In FIG. 2, one such hemodynamical sensor 15 for measuring the left ventricle pressure is shown. Other conceivable sensors include sensors for measuring:
Left atrial pressure (LAP)
Aortic pressure (AoP)
Central venous pressure (CVP
Right atrial pressure (RAP)
Right ventricular pressure (RVP)
Pulmonary artery pressure (PAP)
   Another conceivable hemodynamical signal that may be used is heart sound measured, for example, using a 3D sensor (e.g. accelerometer or microphone) arranged in the can of the cardiac stimulator. This 3D sensor may also be used to determine the body posture. Further, a photoplethysmograph signal (PPG) can be used as hemodynamical index signal. Yet another conceivable hemodynamical signal that may be used is a lead accelerometer signal. Movement and acceleration of heart wall during the heart cycle deliver a continuous signal that may be used for timing optimization. For example, a tip accelerometer may be used for this purpose (Bordachar P., “Hemodynamic assessment of right, left and biventricular pacing by peak endocardial acceleration and echography in patient with end-stage heart-failure”, Pacing Clin Electrophysiol 2000; 23: 1726-30).

With reference now to FIG. 4, a flow chart illustrating steps of a method according to embodiments of the present invention will be discussed.

At step S100, hemodynamical index signals reflecting a mechanical functioning of a heart of a patient are recorded. The hemodynamical index signals are measured by at least one hemodynamical sensor, for example, a left ventricle pressure signal measured by the left ventricle pressure sensor 15 shown in FIG. 2 and/or a cardiogenic impedance signal measured by the impedance measuring module 65 via electrodes of the medical leads 14, 16, 18. The hemodynamical index signals are recorded during measurements sessions at different hemodynamical states, for example, at different AV and/or VV delays. Preferably, a sweep including a number of different AV and/or VV delays according to a predetermined scheme is executed by the timing circuitry 46 using the pacing module 42.

At step S120, corresponding hemodynamical reference signals reflecting a mechanical functioning of the heart measured using non-implantable equipment 3 are recorded. The hemodynamical reference signals are measured during measurement sessions at the corresponding hemodynamical states, for example, at the corresponding AV and/or VV delays during the above mentioned sweep.

The steps S100 and S120, respectively, can be executed simultaneously but are, at least, executed during corresponding hemodynamical states, e.g. during the same AV and/or VV delays and/or at the same posture and/or at the same activity level.

Thereafter, at step S140, at least one hemodynamical index parameter is extracted from the recorded hemodynamical index signals for each hemodynamical state by the calculation module 62, which hemodynamical index parameter is a measure of the mechanical functioning of the heart in a specific hemodynamical state. For example, from a left ventricle pressure signal, LV dP/dtₘₐₓ may be extracted and from cardiogenic impedance signals, for example, peak-to-peak (p2p), slope, fractionation, diastolic dispersion, or average value may be extracted. Further, it is possible to extract or determine, for example, phase angle, imaginary part, or magnitude of the impedance.

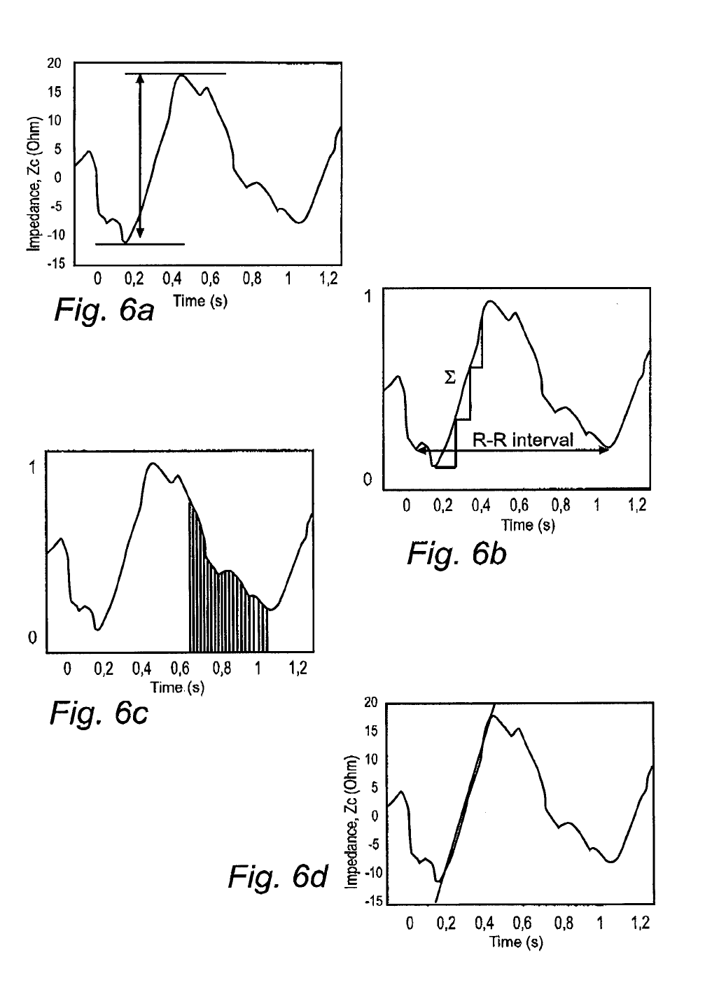

The p2p value can be calculated by taking the maximum value in each heart cycle minus the minimum value in the same heart cycles. In FIG. 6a, an example is illustrated.

With reference to FIG. 6b, the parameter fractionation is calculated for each cycle by first determining the duration of the beat as the R-R interval (i.e. the interval between two successive R events). The amplitude of the impedance signals for this beat is scaled and translated to the interval [0, 1]. The length of the scaled curve is obtained using Pythagoras theorem. Finally, the length of the scaled curve is divided by the duration of the beat to calculate the fractionation.

The parameter diastolic dispersion can be calculated as follows. For each cycle, the duration is obtained by determining the R-R interval. The duration and amplitude of the impedance signal are normalized. Thereafter, the average amplitude during the diastolic phase, [0.6, 1] in FIG. 6c, is calculated to determine the diastolic dispersion.

In order to calculate the parameter slope, the maximum first order derivative of the impedance signal during systole is identified for each beat or cycle. For example, with reference to FIG. 6b, the parameter slope can be calculated for each beat as follows. First, the impedance signal in a time window having a predetermined length, e.g. 350 ms, starting with a predetermined event, e.g. the R-event, is selected for the analysis. The impedance slope is calculated by taking numeral gradient of the time window. A search to identify the longest consecutive data segment having a positive slope is performed. Thereafter, a further search is performed to identify maximum slope of the segment found in the preceding step.

Then, at step S160, a hemodynamical index model is created based on the at least one hemodynamical index parameter and the hemodynamical index model is calibrated by comparisons between output results from the hemodynamical index model and the corresponding hemodynamical reference signals. For example, the model can be created by using multivariate partial least square (PLS) regression based on input from impedance parameters from two measured impedance vectors. For example, a first vector including a current injection between RV tip 28 and a first LV ring 23 and voltage measurement between the same electrodes, and a current injection between RV tip 28 and RV ring 30 and voltage measurement between the same electrodes. In order to assess the results from the hemodynamical model, the produced hemodynamical index is compared against the measured hemodynamical reference signal at the different hemodynamical states at which the signals were recorded, for example, at varying AV and/or VV delays.

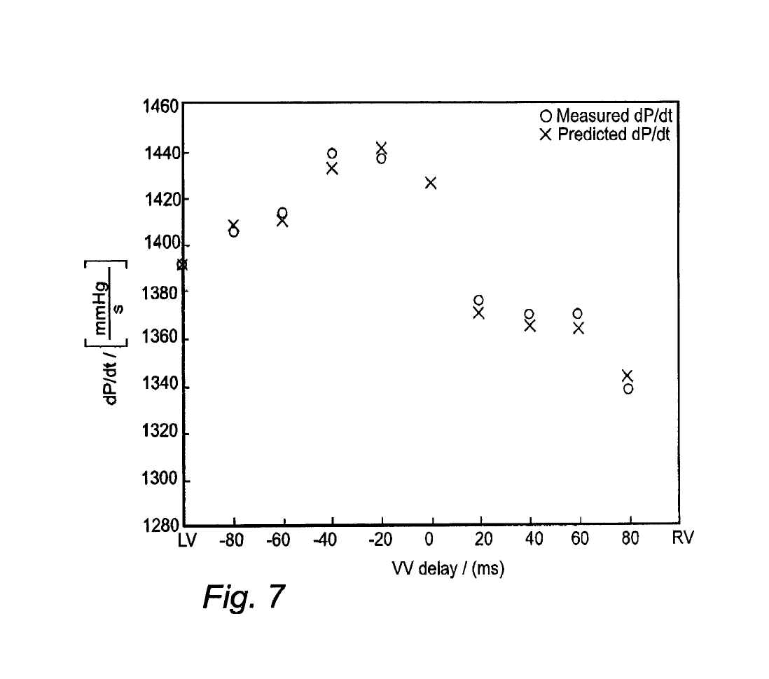

In FIG. 7, an example of a comparison between hemodynamical reference signals and hemodynamic indices is shown. A comparison of LV dP/dtₘₐₓ with the impedance based hemodynamic index plotted against VV delay variation is displayed in FIG. 7.

At step S180, a derived hemodynamical index may be used in determining timing parameter settings of the cardiac stimulator 10. For example, the patient specific hemodynamical index can be used for ambulatory device optimization. According to an embodiment of the present invention, an optimization of timing parameter is performed at scheduled time points or at times triggered by predetermined events, such as detected fusion beats or at a predetermined activity level detected by the accelerometer. For example, for all AV and VV delays at, e.g. +/−10 to 40 ms around the currently programmed delays, the AV and VV delays are programmed according to a predetermined scheme. At each combination a AV and a VV delay, hemodynamical index signals are recorded, e.g. cardiogenic impedance, when steady-state has been reached for that particular hemodynamical state. For each combination, the hemodynamical index parameter (-s) is calculated and a hemodynamical index based on the stored hemodynamical index model is derived. Thereafter, the AV and VV delays corresponding to the highest index value is selected and the cardiac stimulator is programmed with these new timing parameters. Below, with reference to FIG. 8, an optimization procedure in accordance with an embodiment of the present invention will be described.

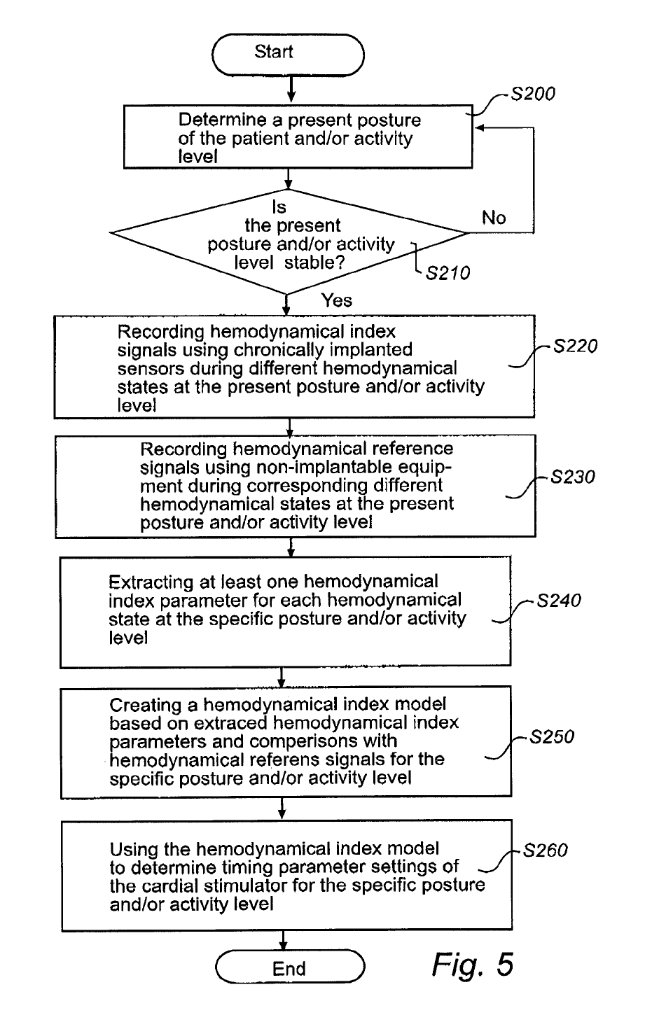

With reference to FIG. 5, a flow chart illustrating another embodiment of the present invention will be discussed. First, at step S200, a present posture of the patient is determined. For example, a 3D sensor (e.g. accelerometer or microphone) arranged in the can of the cardiac stimulator may also be used to determine the body posture of the patient 5. At step S210, it is verified that the posture is stable, for example, the present posture must be prevalent during a predetermined period of time. If it is verified that a present posture is stable, the procedure continuous to step S220. However, if the posture is not stable, the procedure returns to step S200 so as to identify and determine the new posture. This is repeated until a stable posture can be verified.

When it has been verified that the present posture is stable, hemodynamical index signals reflecting a mechanical functioning of a heart of a patient are recorded at step S220. The hemodynamical index signals are measured by at least one hemodynamical sensor, for example, a left ventricle pressure signal measured by the left ventricle pressure sensor shown in FIG. 2 and/or a cardiogenic impedance signal measured by the impedance measuring module 65 via electrodes of the medical leads 14, 16, 18. The hemodynamical index signals are recorded during measurements sessions at different hemodynamical states, for example, at different AV and/or VV delays. Preferably, a sweep including a number of different AV and/or VV delays are performed. The hemodynamical index signals being recorded are tagged with the present posture during which they were recorded. If it is determined that the posture has changed during the measurements of the hemodynamical index signals, the procedure is terminated until that posture has been reached again. Alternatively, the procedure is terminated and returns to step S200 to start again with determining a posture.

At step S230, corresponding hemodynamical reference signals reflecting a mechanical functioning of the heart measured using non-implantable equipment 3 are recorded for the present posture. The hemodynamical reference signals are measured during measurement sessions at the corresponding hemodynamical states, for example, at the corresponding AV and/or VV delays during the above mentioned sweep. The recorded hemodynamical reference signals are tagged with the present posture during which they were recorded. If it is determined that the posture has changed during the measurements of the hemodynamical reference signals, the procedure is terminated until that posture has been reached again. Alternatively, the procedure is terminated and returns to step S200 to start again with determining a posture.

The steps S220 and S230, respectively, can be executed simultaneously but are, at least, executed during corresponding hemodynamical states, e.g. during the same AV and/or VV delays and/or at the same posture and/or at the same activity level.

Thereafter, at step S240, at least one hemodynamical index parameter is extracted from the recorded hemodynamical index signals for each hemodynamical state by the calculation module 62, which hemodynamical index parameter is a measure of the mechanical functioning of the heart in a specific hemodynamical state and the specific posture during which the hemodynamical index and reference signals were obtained. For example, from a left ventricle pressure signal, LV dP/dtₘₐₓ may be extracted and from cardiogenic impedance signals, for example, peak-to-peak (p2p), slope, fractionation, diastolic dispersion, or average value may be extracted.

At step S250, a hemodynamical index model is created for the specific posture based on the at least one hemodynamical index parameter. For example, the model can be created by using multivariate partial least square (PLS) regression based on input from impedance parameters from two measured impedance vectors. So as to assess the results from the hemodynamical model, the derived hemodynamical index is compared with the measured hemodynamical reference signal at the different hemodynamical states at which the signals were recorded, for example, at varying AV and/or VV delays for the particular posture. Since the morphology of the recorded hemodynamical waveforms change with the posture of the patient, the calibration of the hemodynamical index model against recorded hemodynamical reference signals will not be the same for each posture. The process of finding a hemodynamical index model capable of producing an accurate and reliable hemodynamical index must therefore be repeated for every used posture, e.g. supine and standing up.

Thereafter, at step S260, derived hemodynamical index may be used in determining timing parameter settings of the cardiac stimulator 10 for that specific posture. For example, the patient specific hemodynamical index can be used for ambulatory device optimization. According to an embodiment of the present invention, an optimization of timing parameters is performed at scheduled time points or at times triggered by predetermined events, such as detected fusion beats or at a predetermined activity level detected by the accelerometer. The present posture of the patient must also be determined and it must be verified that the posture is stable. If there are several hemodynamical index models for different postures, the hemodynamical index model for the specific present posture is selected. The same posture must be prevalent during the whole optimization process otherwise the optimization procedure is terminated until a stable posture anew is reached. When it has been determined that a present posture is stable a hemodynamical index model adapted for that particular posture is selected and the optimization process can be initiated. For example, for all AV and VV delays at, e.g. +/−10 to 40 ms around the currently programmed delays, the AV and VV delays are programmed according to a predetermined scheme. At each combination of AV and VV delays, hemodynamical index signals are recorded, e.g. cardiogenic impedance, when steady-state has been reached for that particular hemodynamical state. For each combination, the hemodynamical index parameter (−s) is calculated and a hemodynamical index based on the stored hemodynamical index model is derived. Thereafter, the AV and VV delays corresponding to the highest index value is selected and the cardiac stimulator is programmed with these new timing parameters.

The embodiments described with reference to FIG. 4 or 5 can also be combined with a determination of an activity level of the patient. Hence, a hemodynamical index model for a specific posture and/or a specific activity level can be created. For example, in step S200 a present activity level may also be determined and in step S210 is may be checked whether the present activity level stable.

With reference to FIG. 8, an optimization procedure in accordance with embodiments of the present invention will now be described. At step S300, a present posture and/or activity level of the patient may be determined. Then, at step S310, it is checked whether the present posture and/or activity level is stable. These steps may be optional and included if different hemodynamical index models for different postures and/or activity levels have been created, for example, as described above with reference to FIG. 5. The optimization is initiated at step S320, optionally preceded by a determination of posture and/or activity level, by performing a sweep over a predetermined number of different timing parameter settings of the cardiac stimulator 10. Each timing parameter setting results in a specific hemodynamical state of the heart 12. At step S330, at least one hemodynamical index signal is recorded for each timing parameter setting, e.g. each combination of AV and VV delays. Thereafter, at step S340, at least one hemodynamical index parameter is extracted from the recorded at least one hemodynamical index signal for each timing parameter setting and is inputted into the hemodynamical model. At step S350, a hemodynamical index is derived from the hemodynamical index model for each timing parameter setting. Thereafter, at step S360, the timing parameter setting that corresponds to the maximal hemodynamical index for the cardiac stimulator is selected for re-programming of the stimulator.

Although certain embodiments and examples have been described herein, it will be understood by those skilled in the art that many aspects of the devices and methods shown and described in the present disclosure may be differently combined and/or modified to form still further embodiments. Alternative embodiments and/or uses of the devices and methods described above and obvious modifications and equivalents thereof are intended to be within the scope of the present disclosure. Thus, it is intended that the scope of the present invention should not be limited by the particular embodiments described above, but should be determined by a fair reading of the claims that follow.

1. A method for determining cardiac pacing settings of a cardiac stimulator system including a cardiac stimulator, non-implantable equipment, and a number of electrodes and at least one chronically implantable hemodynamical sensor connectable to the cardiac stimulator, the method comprising:
recording hemodynamical index signals reflecting a mechanical functioning of a heart of a patient, wherein the hemodynamical index signals are measured by the at least one hemodynamical sensor and/or electrodes during measurement sessions at different hemodynamical states;
recording corresponding hemodynamical reference signals reflecting a mechanical functioning of the heart measured using the non-implantable equipment, wherein the hemodynamical reference signals are measured during measurement sessions at different hemodynamical states;
extracting at least one hemodynamical index parameter from the recorded hemodynamical index signals for each hemodynamical state, the at least one hemodynamical index parameter being a measure of the mechanical functioning of the heart in a specific hemodynamical state;
creating a hemodynamical index model, wherein the hemodynamical index model is based on the at least one hemodynamical index parameter and comparisons between output results from the hemodynamical index model and corresponding hemodynamical reference signals, wherein a hemodynamical index can be derived from the hemodynamical index model; and
using the hemodynamical index model in determining timing parameter settings of the cardiac stimulator.

2. The method according to claim 1, comprising:
determining a posture of the patient;
verifying that the posture is stable;
recording hemodynamical index signals reflecting a mechanical functioning of a heart of a patient at the posture;
recording corresponding hemodynamical reference signals reflecting a mechanical functioning of the heart at the posture;
extracting at least one hemodynamical index parameter from the recorded hemodynamical index signals for each hemodynamical state when the patient is in the posture;
creating a hemodynamical index model for the posture; and
using the hemodynamical index model in determining cardiac pacing settings of the cardiac stimulator for the posture.

3. The method according to claim 1, comprising:
determining an activity level of the patient;
verifying that the activity level is stable;
recording hemodynamical index signals reflecting a mechanical functioning of the heart of the patient at the activity level;
recording corresponding hemodynamical reference signals reflecting a mechanical functioning of the heart at the activity level;
extracting at least one hemodynamical index parameter from the recorded hemodynamical index signals for each hemodynamical state when the patient is at the activity level;
creating a hemodynamical index model for the activity level; and
using the hemodynamical index in determining cardiac pacing settings of the cardiac stimulator for the activity level.

4. The method according to claim 1, wherein using the hemodynamical index comprises:
performing a sweep over a predetermined number of different timing parameter settings of the cardiac stimulator, each timing parameter setting resulting in a specific hemodynamical state of the heart;
recording at least one hemodynamical index signal for each timing parameter setting;
extracting at least one hemodynamical index parameter from the recorded at least one hemodynamical index signal for each timing parameter setting;
deriving a hemodynamical index using the hemodynamical index model for each timing parameter setting; and
selecting timing parameter setting corresponding to the maximal hemodynamical index for the cardiac stimulator.

5. The method according to claim 4, further comprising:
determining a present posture of the patient;
verifying that the posture is stable;
performing the sweep over a predetermined number of different timing parameter settings of the cardiac stimulator;
recording at least one hemodynamical index signal for each timing parameter setting;
extracting at least one hemodynamical index parameter from the recorded at least one hemodynamical index signal for each timing parameter setting;
selecting a hemodynamical index model for the present posture or adapting the hemodynamical index model with regard to the posture;
deriving a hemodynamical index using the selected hemodynamical index model for each timing parameter setting; and
selecting timing parameter setting corresponding to the maximal hemodynamical index for the cardiac stimulator.

6. The method according to claim 5, further comprising
determining an activity level of the patient;
verifying that the activity level is stable;
performing the sweep over a predetermined number of different timing parameter settings of the cardiac stimulator;
recording at least one hemodynamical index signal for each timing parameter setting;
extracting at least one hemodynamical index parameter from the recorded at least one hemodynamical index signal for each timing parameter setting;
selecting a hemodynamical index model for the present activity level or adapting the hemodynamical index model with regard to the activity level;
deriving a hemodynamical index using the selected hemodynamical index model for each timing parameter setting; and
selecting timing parameter setting corresponding to the maximal hemodynamical index for the cardiac stimulator.

7. The method according claim 1, further comprising:
inducing hemodynamical changes affecting the mechanical functioning of the heart, wherein the different hemodynamical states of the heart are created by altering timing parameter settings of the cardiac stimulator according to a predetermined scheme.

8. The method according to claim 1, wherein recoding hemodynamical index signals includes one or more of the following:
recording hemodynamical pressure signals including any one of left atrial pressure (LAP), left ventricular pressure (LVP), aortic pressure (AoP), central venous pressure (CVP), right atrial pressure (RAP), right ventricular pressure (RVP), or pulmonary artery pressure (PAP); and/or
recording heart sound signals; and/or
recording cardiac impedance waveforms, wherein the impedance waveforms are based on cardiac impedance signals measured by at least one electrode configuration; and/or
recording hemodynamic photoplethysmographic signals; and/or recording accelerometer signals reflecting movement and/or acceleration of heart wall during heart cycles.

9. A cardiac stimulator system having a cardiac stimulator, non-implantable equipment, and a number of electrodes and at least one chronically implantable hemodynamical sensor connectable to the cardiac stimulator, the system comprising:
a data collection module of the cardiac stimulator is configured to collect and record hemodynamical index signals reflecting a mechanical functioning of a heart of a patient, wherein the hemodynamical index signals are measured by the at least one hemodynamical sensor and/electrodes during measurement sessions at different hemodynamical states;
the non-implantable equipment is configured to record corresponding hemodynamical reference signals reflecting a mechanical functioning of the heart measured using the non-implantable equipment, wherein the hemodynamical reference signals are measured during measurement sessions at different hemodynamical states;
a calculation module configured to:
receive the recorded hemodynamical index signals and the recorded hemodynamical reference signals;
extract at least one hemodynamical index parameter from the recorded hemodynamical index signals for each hemodynamical state, the at least one hemodynamical index parameter being a measure of the mechanical functioning of the heart in a specific hemodynamical state; and
create a hemodynamical index model, wherein the hemodynamical index model is based on the at least one hemodynamical index parameter and comparisons between output results from the hemodynamical index model and corresponding hemodynamical reference signals, wherein a hemodynamical index can be derived from the hemodynamical index model; and
an optimization module is configured to use the hemodynamical index model in determining timing parameter settings of the cardiac stimulator.

10. The system according to claim 9, wherein the calculation module and the optimization module is arranged in the cardiac stimulator.

11. The system according to claim 9, wherein the system comprises:
a controller configured to determine a posture of the patient and verify that the posture is stable using input from a posture sensor; wherein
the data collection module is configured to record hemodynamical index signals reflecting a mechanical functioning of a heart of a patient at the posture;
the non-implantable equipment is configured to recording corresponding hemodynamical reference signals reflecting a mechanical functioning of the heart at the posture;
the calculation module is configured to extract at least one hemodynamical index parameter from the recorded hemodynamical index signals for each hemodynamical state when the patient is in the posture;
the calculation module is configured to create a hemodynamical index model for the posture; and
the optimization module is configured to use the hemodynamical index model in determining cardiac pacing settings of the cardiac stimulator for cad the posture.

12. The system according to claim 9, comprising:
a controller configured to determine an activity level of the patient and verify that the activity level is stable based on input from an activity detector; wherein
the data collection module is configured to record hemodynamical index signals reflecting a mechanical functioning of a heart of a patient at the activity level;
the non-implantable equipment is configured to record corresponding hemodynamical reference signals reflecting a mechanical functioning of the heart at the activity level;
the calculation module is configured to extract at least one hemodynamical index parameter from the recorded hemodynamical index signals for each hemodynamical state when the patient is at the activity level;
the calculation module is configured to create a hemodynamical index model for the activity level; and
the optimization module is configured to use the hemodynamical index in determining cardiac pacing settings of the cardiac stimulator for the activity level.

13. The system according to claim 9, wherein the optimization module is configured to, in an optimization procedure:
instruct the timing circuitry to execute a sweep over a predetermined number of different timing parameter settings of the cardiac stimulator, each timing parameter setting resulting in a specific hemodynamical state of the heart;
instruct the data collection module to record at least one hemodynamical index signal for each timing parameter setting;
instruct the calculation module to extract at least one hemodynamical index parameter from the recorded at least one hemodynamical index signal for each timing parameter setting;
derive a hemodynamical index using the hemodynamical index model for each timing parameter setting; and
select timing parameter setting corresponding to the maximal hemodynamical index for the cardiac stimulator.

14. The system according to claim 13, wherein the controller is configured to:
determine a present posture of the patient and verify that the posture is stable; and
inform the optimization module of a present posture;
wherein the optimization module is configured to select a hemodynamical model adapted for the present posture for an optimization module.

15. The system according to claim 14, wherein
the controller is configured to:
determine a present activity level of the patient and that the activity level is stable; and
inform the optimization module of a present activity level; and
wherein the optimization module is configured to select a hemodynamical index model adapted to the activity level for an optimization procedure.

16. The system according to claim 9, wherein a timing circuitry is configured to alter timing parameter settings of the cardiac stimulator according to a predetermined scheme to induce hemodynamical changes affecting the mechanical functioning of the heart.

17. The system according to claim 9, wherein the data collection module which is configured to collect and record hemodynamical index signals includes one or more of the following:
hemodynamical pressure signals including any one of left atrial pressure (LAP), left ventricular pressure (LVP), aortic pressure (AoP), central venous pressure (CVP), right atrial pressure (RAP), right ventricular pressure (RVP), or pulmonary artery pressure (PAP); and/or
heart sound signals; and/or
cardiac impedance waveforms, wherein the impedance waveforms are based on cardiac impedance signals measured by at least one electrode configuration; and/or
hemodynamic photoplethysmographic signals; and/or
recording accelerometer signals reflecting movement and/or acceleration of heart wall during heart cycles.
